(19) United States
(12) Patent Application Publication
(10) Pub. No.: US 2026/0355542 A1
(43) Pub. Date: Nov. 12, 2026

(54) OPTICAL TRACKING SYSTEMS AND METHODS

(71) Applicant: HISTOSONICS, INC., Ann Arbor, MI (US)

(72) Inventors: Thaddeus Hoffman, Brooklyn Park, MN (US); Shreyas Shrinivas Kallapur, Minneapolis, MN (US); Evan McNamara, Minneapolis, MN (US); Bret McNamara, Madison, WI (US); Matthew A. Rootes, Plymouth, MN (US)

(73) Assignee: HISTOSONICS, INC., Ann Arbor, MI (US)

(21) Appl. No.: 19/672,088

(22) Filed: May 8, 2026

Related U.S. Application Data
(60) Provisional application No. 63/803,265, filed on May 9, 2025.

(51) Int. Cl. A61N 7/00

(52) U.S. Cl. CPC A61N 7/00; A61N 2007/0039; A61N 2007/0052

(57) ABSTRACT

A histotripsy system including a cone beam computed tomography (CBCT) imaging system including a first optical tracking marker, a robotically driven therapy transducer including a second optical tracking marker, an operating table configured to support a patient and including a third optical tracking marker, a plurality of cameras configured to emit light and receive reflected light from the first second and third optical tracking markers, and a computing device including a memory and a processor, the memory storing therein an application which when executed by the processor cause the computing device to receive from the plurality of cameras signals representative of the reflected light, determine a position and orientation of the first, second, and third optical tracking marker, and register the CBCT imaging system, the therapy transducer, and the operating table to one another in operating room coordinates.

### FIELD

This disclosure is directed to high intensity therapeutic ultrasound (HITU) systems configured to produce acoustic cavitation, methods, devices and procedures for the minimally and non-invasive treatment of healthy, diseased and/or injured tissue. In particular, the disclosure is directed to systems and methods of registering and maintaining registration of components of the HITU system, intraprocedural images, and three-dimensional models generated from the images.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a histotripsy system including a cone beam computed tomography (CBCT) imaging system including a first optical tracking marker; a robotically driven therapy transducer including a second optical tracking marker; an operating table configured to support a patient and including a third optical tracking marker; a plurality of cameras configured to emit light and receive reflected light from the first second and third optical tracking markers; and a computing device including a memory and a processor, the memory storing therein an application which when executed by the processor cause the computing device to: receive from the plurality of cameras signals representative of the reflected light; determine a position and orientation of the first, second, and third optical tracking marker; and register the CBCT imaging system, the therapy transducer, and the operating table to one another in operating room coordinates. Other embodiments of this aspect include corresponding computer systems, apparatus, and computer programs recorded on one or more computer storage devices, each configured to perform the actions of the methods.

Implementations may include one or more of the following features. The histotripsy system where the application when executed by the processor, causes the computing device to determine a position and orientation of the CBCT imaging system in operating room coordinates while the CBCT imaging system is capturing images of the patient. CBCT images are captured at imaging coordinates and the application when executed by the processor, causes the computing device to register the captured CBCT images to the operating room coordinates. The application when executed by the processor cause the computing device to display the CBCT images or a three-dimensional (3d) model in a user interface. The application when executed by the processor cause the computing device to define a target for therapy in the CBCT images or 3D model. The therapy transducer includes imaging functionality, and where the application when executed by the processor cause the computing device to drive the robotically driven therapy transducer to axial ends of the defined target to image the target within the patient. The application when executed by the processor cause the computing device to confirm registration of the CBCT images or 3D model with the operating room coordinates. The histotripsy system may include driving the therapy transducer and delivering histotripsy therapy to the target. The application when executed by the processor cause the computing device to detect movement of the CBCT imaging system, the therapy transducer, or operating bed and updates the registration. The first and third optical tracking maker including a spherical shell. The spherical shell includes a plurality of protrusions configured to receive a passive fiducial. The first and third optical tracking markers include a marker insert configured to mate with the spherical shell. The histotripsy system may include a marker base, the marker base including a slot configured to receive a rail of the marker insert. The marker insert includes a flexible tab configured to mate with a catch on the marker base to secure the marker insert and attached spherical shell to the marker base. The marker base includes a rotational adjustment mount, the rotational adjustment mount enabling movement of the optical tracking markers relative to the CBCT imaging system or operating table to ensure visibility of at least a threshold value of the passive fiducials by each of the plurality of cameras. The second optical tracking marker mates with the therapy transducer without impacting the therapy transducer’s range of motion. The second optical tracking marker includes a plurality of protrusions configured to receive a passive fiducial. The second optical tracking marker may include a cable management feature. Implementations of the described techniques may include hardware, a method or process, or computer software on a computer-accessible medium.

A further general aspect of the disclosure is an optical tracking marker including a spherical shell having a plurality of protrusions configured to receive a passive fiducial; a marker insert configured to mate with the spherical shell; and a marker base, the marker base including a slot configured to receive a rail of the marker insert and a catch configured to mate with a flexible tab on the marker insert to secure the marker insert and attached spherical shell to the marker base.

Yet a further general aspect of the disclosure is an optical tracking marker having a shell formed to mate with a histotripsy therapy transducer, the shell including a plurality of protrusions configured to receive a passive fiducial; and a cable management feature configured to receive cables of the histotripsy therapy transducer and prevent the cables from obscuring the passive fiducial.

### BRIEF DESCRIPTION OF THE DRAWINGS

The features of the disclosure are set forth with particularity in the claims that follow. A better understanding of the features and advantages of the disclosure will be obtained by reference to the following detailed description that sets forth illustrative embodiments, in which the principles of the disclosure are utilized, and the accompanying drawings of which:

FIG. 1 is a perspective view of a histotripsy system in accordance with the disclosure;

FIG. 2 depicts a method of registration of the components of the histotripsy system in accordance with the disclosure;

FIG. 3 is a perspective view of an optical tracking marker in accordance with the disclosure;

FIG. 4 is a cross-sectional view of the optical tracking marker of FIG. 3, in accordance with the disclosure;

FIG. 5 is a rear view of the optical tracking marker of FIG. 3, in accordance with the disclosure;

FIG. 6 is a perspective view of a marker insert in accordance with the disclosure;

FIGS. 7A and 7B are perspective views of a marker base in accordance with the disclosure;

FIG. 8 is a perspective view of an optical tracking marker connected to a therapy transducer in accordance with the disclosure;

FIG. 9 is a front view of the optical tracking marker of FIG. 8 connected to a therapy transducer in accordance with the disclosure; and

FIG. 10 is a rear perspective view of the optical tracking marker of FIG. 8 connected to a therapy transducer in accordance with the disclosure.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing, and ablation. Furthermore, due to tissue selective properties, histotripsy may be used to create a cytoskeleton that allows for subsequent tissue regeneration either de novo or through the application of stem cells and other adjuvants. Histotripsy can also be used to cause the release of delivered agents such as chemotherapy and immunotherapy by locally causing the release of these agents by the application of acoustic energy to the targets. As will be described below, the acoustic cavitation system may include various sub-systems, including a cart, therapy, integrated imaging, robotics, coupling and software subsystems. The acoustic cavitation system also may comprise various other components, ancillaries and accessories, including but not limited to computers, processors, memory, software, applications, cables, connectors, networking devices, power supplies, displays, drawers/storage, doors, wheels, and various simulation and training tools, etc. All systems, methods and means creating/controlling/delivering histotripsy are considered to be a part of this disclosure.

### HISTOTRIPSY

Histotripsy is achieved by generating short, high amplitude, focused ultrasound pulses to generate a dense, energetic, “bubble cloud,” capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically affect tissue structure, and in some cases liquefy, suspend, solubilize and/or destruct tissue into sub-cellular components.

Histotripsy can be applied in various forms, including: 1) intrinsic-threshold histotripsy which delivers pulses typically with a 1-2 cycles of high amplitude negative/tensile phase pressure exceeding the intrinsic threshold to generate cavitation in the medium (e.g., ~24-28 MPa for water-based soft tissue), 2) shock-scattering histotripsy which delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) boiling histotripsy which employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism.”

### THE SYSTEM

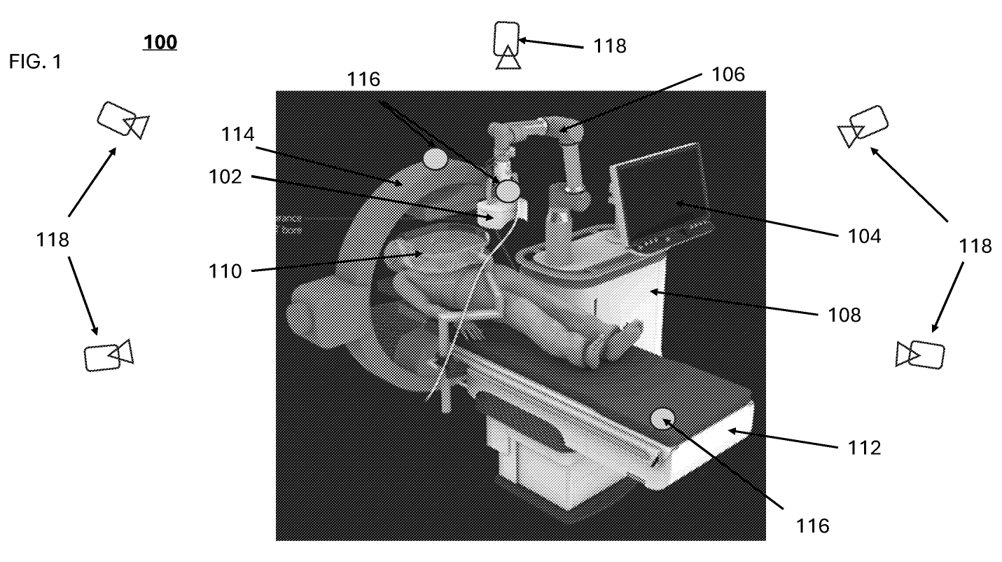

FIG. 1 depicts a histotripsy system 100 in accordance with the disclosure. The histotripsy system 100 includes a HITU therapy and imaging transducer 102, a display and control panel 104, a robotic positioning arm 106, and a cart 108. The system further includes an ultrasound coupling interface 110 including a source of coupling medium (not shown but typically associated with the cart 108). A patient P is positioned on an operating table 112, with the coupling interface 110 placed on the patient. To acoustically couple the therapy and imaging transducer 102 with the patient, the therapy and imaging transducer 102 is placed within the coupling interface 110 and any air bubbles removed.

The cart 108 may enclose, one or more of various sub-systems, including a therapy sub-system that can create, apply, focus and deliver acoustic cavitation/histotripsy through one or more therapy and imaging transducers 102, an integrated imaging sub-system (or connectivity thereto) allowing real-time visualization and display of the treatment site and histotripsy effect through-out the procedure (e.g., via, a robotics positioning sub-system to mechanically and/or electronically steer the therapy and imaging transducer 102) and further enabled to connect/support or interact with a coupling sub-system to allow acoustic coupling between the therapy and imaging transducer 102 and the patient, and software to communicate, control and interface with the system and computer-based control systems (and other external systems) and various other components, ancillaries and accessories, including one or more user interfaces and displays, and related guided work-flows, all working in part or together. The histotripsy system 100 may further comprise various fluidics and fluid management components, including but not limited to, pumps, valve and flow controls, temperature and degassing controls, and irrigation and aspiration capabilities, as well as providing and storing fluids. It may also contain various power supplies and protectors.

A cone-beam computed tomography (CBCT) imaging system 114 is employed to collect CBCT images of the patient. The images acquired by the CBCT imaging system 114 are received and stored in a memory (e.g., an electronic medical record (EMR)), and made accessible to the control panel 104. The control panel 104, as noted above, includes a memory storing therein one or more applications that when executed by the control panel’s processors enable the accessing of CBCT images of the patient, the identification of one or more targets for therapy, and the generation of 3D models of the patient’s anatomy from the CBCT images.

As will be appreciated, the CBCT imaging system 114, when positioned about the patient P and the coupling interface 110, interferes with the positioning and movement of the therapy and imaging transducer 102 on the robotic positioning arm 106. Thus, assuming the operating table 112 is not moved during a procedure, one or more of the CBCT imaging system 114, the therapy and imaging transducer 102 on the robotic positioning arm 106, must be moved relative to each other to accommodate their respective use.

To enable use of CBCT images, an image coordinate system must be registered to the coordinate system of the operating room (i.e., the therapy and imaging transducer 102). Without such registration, the therapy and imaging transducer 102 cannot be accurately driven to positions and orientation to apply HITU to the targets identified in the CBCT images of the patient. Though the imaging aspects of the therapy and imaging transducer 102 can still be employed to identify the targets within ultrasound images of the patient to drive the therapy and imaging transducer 102 and robotic positioning arm 106, features such as image fusion (e.g., combining CBCT and US images) are not available with registration and maintenance of the registration throughout the procedure.

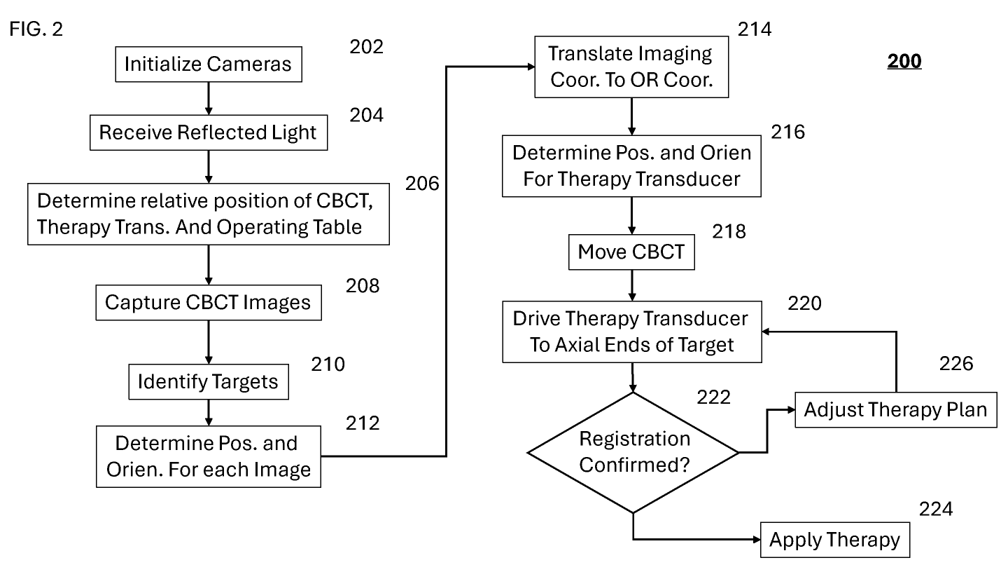

In accordance with the disclosure, to achieve the registration and to maintain the registration each of the CBCT imagine system 114, the imaging and therapy transducer 102, and the operating table 112 each include an optical tracking marker 116. Each optical tracking marker 116 is imaged by one or more cameras 118 in the operating room. The cameras may operate at any of a variety of imaging frequencies including near-infrared (NIR), infrared, visible light, and others. Each camera 118 includes a light source (not shown) that generates and transmits light in the direction of the optical tracking markers 116. Each optical tracking marker 116 includes a plurality of passive fiducials (See. FIG. 2) (e.g., reflectors) that reflect the light transmitted from the light source associated with each camera. The reflected light is received by the cameras 118. The cameras 118 are in communication with a computing device (e.g., in the cart 108) including an application that determines a position and orientation of each tracking marker 116 and the relative position of each tracking marker 116. With the relative position and orientation of the tracking markers 116 determined and the CBCT imaging system 114, the therapy and imaging transducer 102, and the operating table 112, the three components are registered to one another.

As depicted in FIG. 1 multiple cameras 118 may be employed, each camera 118 visualizes (receives the reflected light from) the tracking markers 116, and the relative positions of the tracking markers 116 can be separately determined for each camera 118. These separately determined positions and orientations of the tracking markers 116 can be compared to one another to confirm the relative position and orientation of each tracking marker 116.

With the CBCT imaging system 114 registered to the therapy and imaging transducer 102 and to the operating table 112 movements of any of these components are tracked based on detected changes in the reflected light received by the cameras 118. These changes can then be utilized to maintain the registration.

In accordance with the disclosure, an exemplary procedure is described in connection with FIG. 2 depicting a method 200. At step 202 the cameras 118 initialized and the light emitters begin transmitting light. At step 204 the cameras 118 receive the light reflected by the tracking markers 116 and transmits signals representative of the reflected light are transmitted to the computing device (e.g., on cart 108). At step 206 an application stored in the memory on the computing device when executed by the processor analyzes the signals representative of the received reflect light and determines the relative positions and orientations of each of the CBCT imaging system 114, the operating table 112, and the therapy and imaging transducer 102. At step 208 the CBCT imaging system 114 is utilized to capture a CBCT image data set of the patient. An application on control panel 104 enables presentation of the CBCT images in a user interface at step 210 and functionality of the application allows for identification of one or more targets in the images (either manually or automatically) and a volume to receive histotripsy therapy, thus defining a therapy plan.

During the capture of the CBCT images, a position and orientation of the CBCT imaging system 114 at which each of the images is acquired is determined at step 212. As noted above, the CBCT imaging system 114 defines its own imaging coordinate system. Thus, knowing the position and orientation of the CBCT imaging system 114 at which each image is acquired enables translation of the imaging coordinate system to the operating room coordinate system at step 214. At step 216, the application on the computing device determines a series of locations and orientations (in operating room coordinates) to which the therapy and imaging transducer 102 must be driven to apply therapy to the target, which was originally defined in imaging coordinates. At step 218 the CBCT imaging system 114 is moved away from the patient, and at step 220, the robotic positioning arm 106 is driven to a series of locations in operating room coordinates to position and orient the therapy and imaging transducer 102 to axial extreme ends of the defined target. This serves multiple purposes, first enable ultrasound visualization of the target (e.g., via the therapy and imaging transducer 102), second confirm the registration of the two coordinate systems, and third, to confirm that there are no interferences between the therapy and imaging transducer 102 and the coupling interface 110. If the registration is confirmed at step 222 and there are no interferences the method proceeds to the application of therapy at step 224. However, if the registration is inaccurate, or there are interferences between the therapy and imaging transducer 102 and the coupling interface 110 adjustments to the therapy plan are undertaken at step 226 and the method return to step 220.

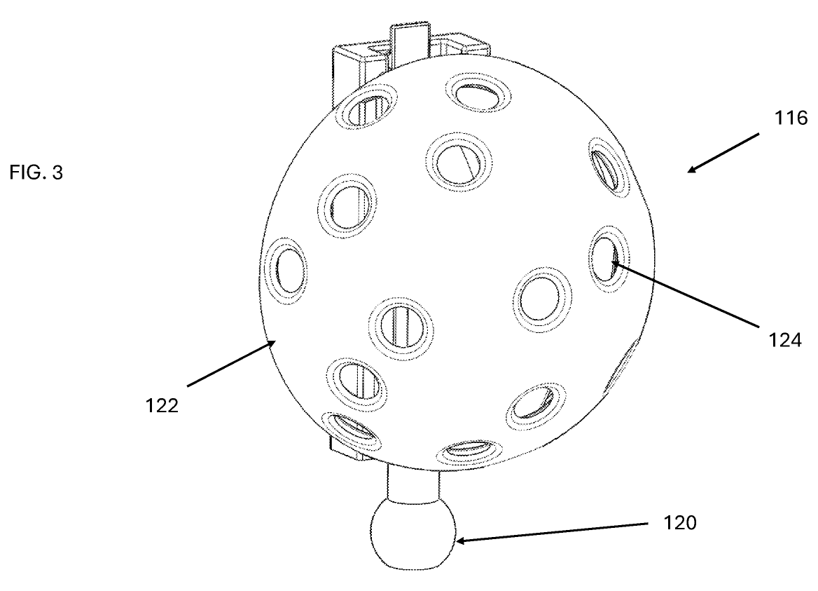

Though the preceding focused primarily on the system and method, the disclosure is not so limited. FIGS. 3-7B depict an optical tracking marker 116 in accordance with the disclosure. Specifically, the optical tracking marker 116 depicted in FIGS. 3-7B is for mounting and securing to CBCT imaging system 114. As noted above, the histotripsy system 100 may include separate tracking markers 116 to enable the real-time tracking of multiple histotripsy system 100 devices (e.g., the CBCT imaging system 114, the operating bed 112, and the therapy and imaging transducer 102). The relative position of these tracking markers 116 (and their related components) is tracked in real time by the external tracking system (e.g., cameras 118 and software applications).

### THE MARKERS

In FIG. 3 the tracking marker 116 includes a rotational adjustment mount 120 (e.g., a ball joint) that enables controlled rotational adjustment of the position and orientation of the tracking marker 116. This ensures that the optical tracking marker 116 is sufficiently visible to each or a threshold number of the cameras 118 in the operating room. The rotational adjustment mount 120 ensures that the tracking marker 116 can be oriented as needed to maintain continuous line-of-sight with the cameras 118, regardless of the surrounding equipment configuration or camera positioning. By allowing for precise repositioning, the rotational adjustment mount 120 guarantees optimal visibility of the tracking marker 116 from multiple angles and degrees of rotation, thereby enhancing the accuracy and reliability of spatial tracking throughout the procedure (and overcome limitations/challenges induced by patient positioning, potential physical interactions between system components and/or the patient (e.g., therapy and imaging transducer 102 collision with the coupling interface), as well as how the various components of the physical components may have obstructed views when optimally positioned for treatment if additional degrees of freedom did not exist). The rotational adjustment mount 120 mates with a socket component (not shown) that attaches to the CBCT imaging system 114 or the operating table 112.

Further, distributed across the external surface of the spherical body 122 of the tracking marker 116 are a plurality of uniformly sized and radially distributed protrusions 124. In one embodiment, each protrusions may be approximately 11 millimeters in diameter and 2mm in height. These protrusions 124 are configured to receive passive fiducials (not shown). The passive fiducials are formed of a reflective material formed as a circular flat disc configured to be press fit into the protrusions 124. Each protrusion 124 is geometrically configured such that its tangent point of an installed passive fiducial aligns with the tangent of the underlying surface of the spherical body 122. This ensures that the passive fiducials maintain a consistent profile with respect to the spherical curvature of the spherical body 122, thereby enabling consistent and reliable detection by optical sensors (e.g., cameras 118) regardless of the CBCT imaging system’s position and orientation. To ensure accurate location and orientation determinations the placement of the protrusions 124, and thus the passive fiducials is configured to maintain at-least two passive fiducial for each tracking marker 116 within the field of view of the cameras 118 throughout the range of motion of the components to which the tracking makers 116 are connected.

The arrangement of the protrusions 124 is optimized to ensure omnidirectional visibility from multiple camera angles without occlusion. The spherical geometry coupled with tangent-aligned passive fiducials provides a highly robust optical signature for multi-camera motion tracking environments as described above in connection with FIG. 1.

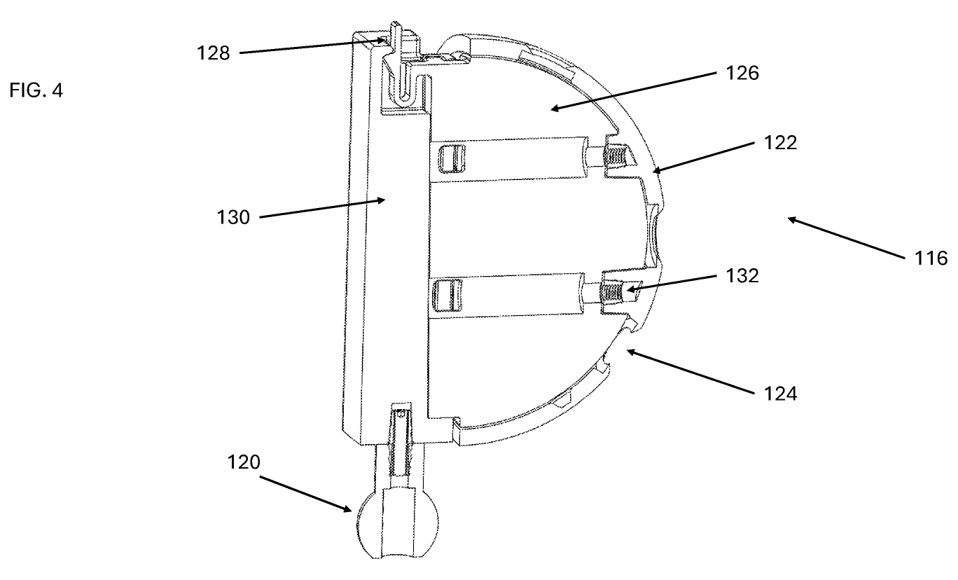

FIG. 4 depicts a cross-sectional view of the tracking marker 116. In FIG. 4 a marker insert 126 is screwed to a back surface of the spherical body 122. The insert 126 and includes a flexible tab 128 configured to secure the tracking marker 116 to a marker base 130. At the opposite end of the marker base 130 configured to receive the flexible tab 128 is the rotational adjustment mount 120.

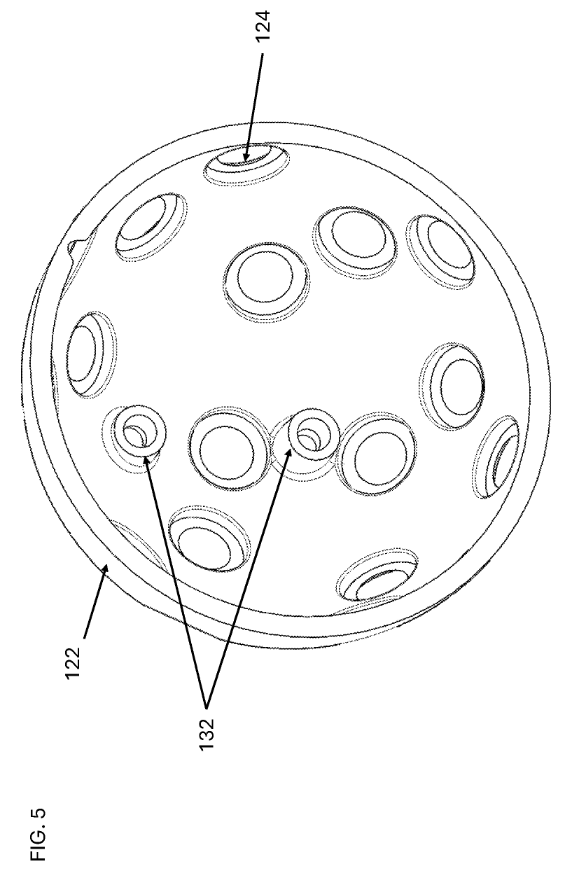

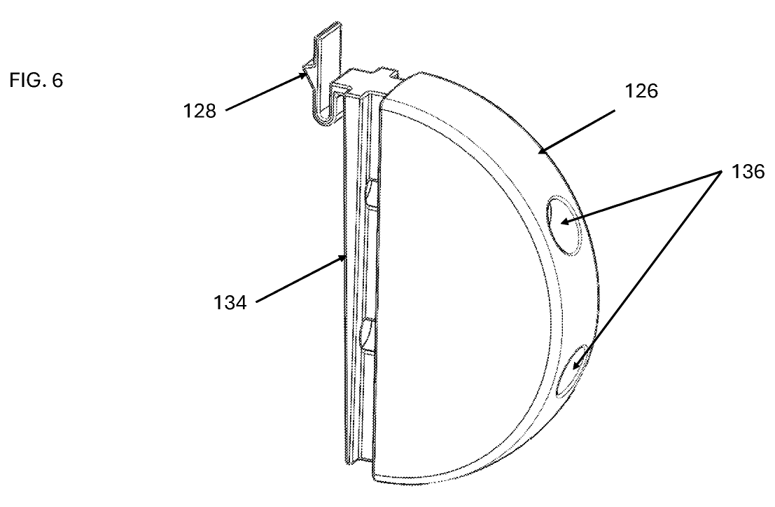

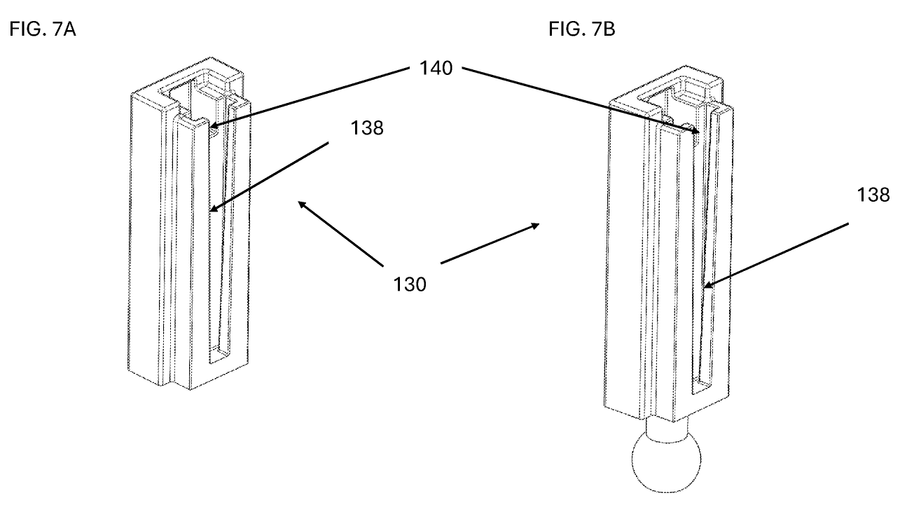

FIG. 5 depicts an internal view of the tracking marker 116. Threaded elements 132 are configured to receive machine screws, or another attachment mean, to connect the spherical body 122 to the marker insert 126. FIG. 6 depicts the marker insert 126 including flexible tab 128. Also depicted in FIG. 6 is a rail 134 configured to mate with a slot 138 formed in the marker base 130 (See FIGS. 7A and 7B). Attachment openings 136 receive the threaded elements 132 and align the spherical body 122 with the marker insert 126.

FIGS. 7A and 7B depict two versions of the marker base 130. Each marker base 130 includes a slot 138 configured to receive the rail of the marker insert 126. A catch 140 is configured to receive the flexible tab 128. As can be seen in FIG. 7A the marker base 130 does not include the rotational adjustment mount 120, while the marker base 130 of FIG. 7B does. The adjustable mount 120 may be removable.

The tracking marker 116 of FIGS. 3-7B may be secured via the marker base 130 to the CBCT imaging system 114 and to the operating table 112. The marker base 130 may be removably mounted (e.g., via a removable pressure sensitive adhesive).

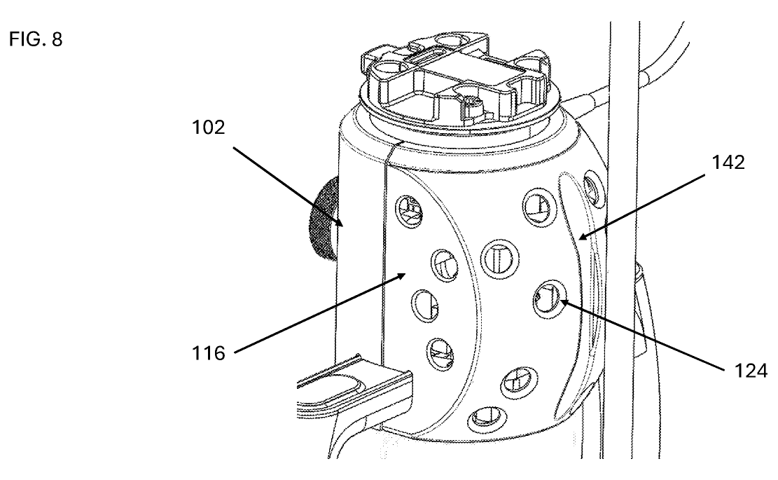

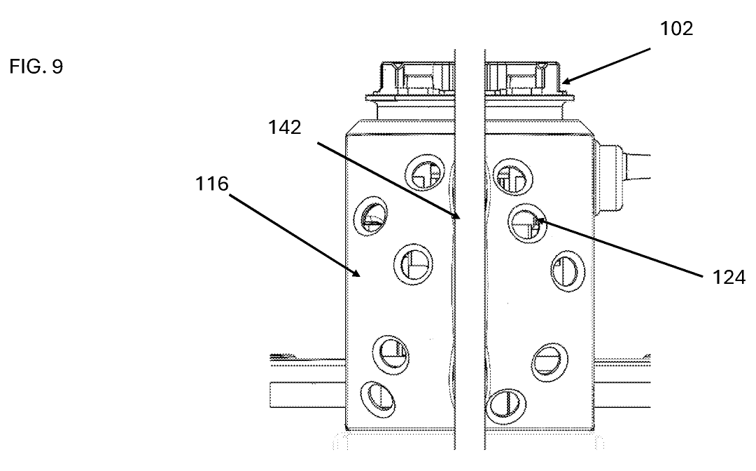

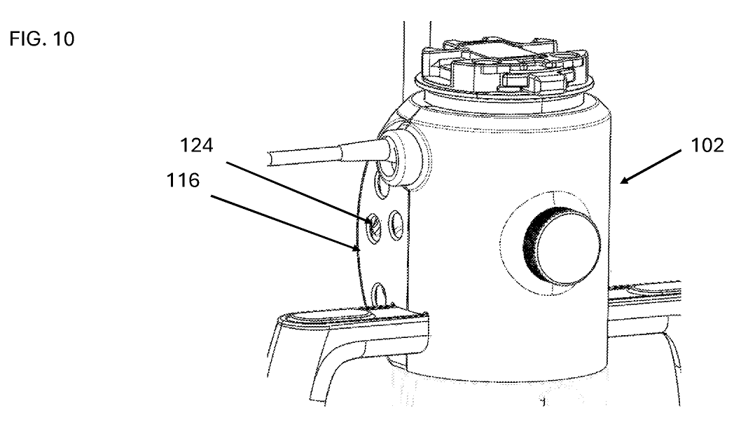

FIGS. 8-10 depict a tracking marker 116 configured to be mounted to the therapy and imaging transducer 102. The tracking marker 116 mounted to the therapy and imaging transducer 102 is designed to house passive fiducials such that the cameras 118 of the histotripsy system 100 can identify the tracking marker 116 with a wide working angle with the design accounting for other critical therapy and imaging transducer features and functionality and potential objects in the field of view of the cameras 118.

As depicted in FIGS. 8-10 the tracking marker 116 is configured not to impede any core functionality of the therapy and imaging transducer 102 or robotic positioning arm 106 including translation and rotation features and cable management, as well as ensuring tracking marker 116 does not increase likelihood of a with the coupling interface or patient. Further the tracking marker 116 is configured not to limit working ranges of the histotripsy system 100.

The tracking marker 116 includes a cable management feature 142 configured to ensure that the ensure that a transducer cable does not occlude the line of sight of the fiducials with the cameras 118. The cable management feature 142 may include an interference fit for installation but has enough space within to allow for free translation of the cable within accounting for the movement of the cable during procedure as the therapy and imaging transducer 102 is moved to apply histotripsy therapy to the target. Further, the cable management feature 142 may accommodate strain relief for the cables of the therapy and imaging transducer 102.

In accordance with aspects of the disclosure, the tracking marker 116 may be designed such that the fiducial placements do not disturb the existing assembly procedure. Further, the planes of passive fiducials (e.g., angle or rotation) may be specifically placed to accommodate for the known working range of the therapy and imaging transducer 102 and the robotic positioning arm 106 during a histotripsy procedure to ensure they are tracked by the cameras 118.

The radius of the tracking marker 116 may be designed to ensure the internal packaging is not disturbed and to enable accessibility to large variation of plane angles. Further, the visible reflective surface of the passive fiducial may be controlled by maintaining the diameter and location of the protrusions on the spherical body 122 of the tracking marker 116.

Though shown in a particular configuration in FIGS. 8-10, the location and size of this feature may be specifically configured to accommodate and intended application/ method of use (e.g., abdominal versus pelvic, etc.) and therapies.

Each of the tracking markers 116 described herein may have distinct passive fiducial placements to ensure easy differentiation of each of the tracking markers 116 during use. This enhances the precise tracking of the positions of the CBCT imaging system 114, the therapy and imaging transducer 102 and the operating table 112, in space. The separate tracking enables use cases where the relative positions of the system components or end-effectors may be moved significantly during the procedure (e.g., CBCT imaging system 114 moving to accommodate patient access or specific target approaches for the therapy and imaging transducer 102).

The tracking markers 116 may be constructed from a range of rigid polymers or lightweight composite materials compatible with additive manufacturing techniques such as stereolithography (SLA) or selective laser sintering (SLS), allowing for customization of the protrusion array density and central slot geometry.

As described herein above, the histotripsy system 100 employing the tracking markers 116 can be moved about the operating room to accommodate a variety of needs (e.g., patient positioning and initial system set-up). The optical tracking system of this disclosure enables the relative positions of the components of the histotripsy system 100 to be optically tracked during the procedure to maintain the registration of the operating room coordinate system with the image coordinate system. In addition, the tracking system enables the determination that elements of the histotripsy system 100 have been bumped unintentionally and can account for those detected changes without requiring a re-registration process.

The embodiments described above are cited by way of example, and that the present invention is not limited to what has been particularly shown and described hereinabove. Rather, the scope of this disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are considered an integral part of the application.

We claim:

1. A histotripsy system comprising:
a cone beam computed tomography (CBCT) imaging system including a first optical tracking marker;
a robotically driven therapy transducer including a second optical tracking marker;
an operating table configured to support a patient and including a third optical tracking marker;
a plurality of cameras configured to emit light and receive reflected light from the first second and third optical tracking markers; and
a computing device including a memory and a processor, the memory storing therein an application which when executed by the processor cause the computing device to:
receive from the plurality of cameras signals representative of the reflected light;
determine a position and orientation of the first, second, and third optical tracking marker; and
register the CBCT imaging system, the therapy transducer, and the operating table to one another in operating room coordinates.

2. The histotripsy system of claim 1, wherein the application that when executed by the processor, causes the computing device to determine a position and orientation of the CBCT imaging system in operating room coordinates while the CBCT imaging system is capturing images of the patient.

3. The histotripsy system of claim 2, wherein CBCT images are captured at imaging coordinates and the application when executed by the processor, causes the computing device to register the captured CBCT images to the operating room coordinates.

4. The histotripsy system of claim 3, wherein the application when executed by the processor cause the computing device to display the CBCT images or a three-dimensional (3D) model in a user interface.

5. The histotripsy system of claim 4, wherein the application when executed by the processor cause the computing device to define a target for therapy in the CBCT images or 3D model.

6. The histotripsy system of claim 5, wherein the therapy transducer includes imaging functionality, and wherein the application when executed by the processor cause the computing device to drive the robotically driven therapy transducer to axial ends of the defined target to image the target within the patient.

7. The histotripsy system of claim 6, wherein the application when executed by the processor cause the computing device to confirm registration of the CBCT images or 3D model with the operating room coordinates.

8. The histotripsy system of claim 7, further comprising driving the therapy transducer and delivering histotripsy therapy to the target.

9. The histotripsy system of claim 1, wherein the application when executed by the processor cause the computing device to detect movement of the CBCT imaging system, the therapy transducer, or operating bed and updates the registration.

10. The histotripsy system of claim 1, wherein the first and third optical tracking maker including a spherical shell.

11. The histotripsy system of claim 10, wherein the spherical shell includes a plurality of protrusions configured to receive a passive fiducial.

12. The histotripsy system of claim 11, wherein the first and third optical tracking markers include a marker insert configured to mate with the spherical shell.

13. The histotripsy system of claim 12, further comprising a marker base, the marker base including a slot configured to receive a rail of the marker insert.

14. The histotripsy system of claim 13, wherein the marker insert includes a flexible tab configured to mate with a catch on the marker base to secure the marker insert and attached spherical shell to the marker base.

15. The histotripsy system of claim 13, wherein the marker base includes a rotational adjustment mount, the rotational adjustment mount enabling movement of the optical tracking markers relative to the CBCT imaging system or operating table to ensure visibility of at least a threshold value of the passive fiducials by each of the plurality of cameras.

16. The histotripsy system of claim 1, wherein the second optical tracking marker mates with the therapy transducer without impacting the therapy transducer’s range of motion.

17. The histotripsy system of claim 16, wherein the second optical tracking marker includes a plurality of protrusions configured to receive a passive fiducial.

18. The histotripsy system of claim 17, wherein the second optical tracking marker further comprises a cable management feature.

19. An optical tracking marker comprising:
a spherical shell including a plurality of protrusions configured to receive a passive fiducial;
a marker insert configured to mate with the spherical shell; and
a marker base, the marker base including a slot configured to receive a rail of the marker insert and a catch configured to mate with a flexible tab on the marker insert to secure the marker insert and attached spherical shell to the marker base.

20. An optical tracking marker comprising:
a shell formed to mate with a histotripsy therapy transducer, the shell including a plurality of protrusions configured to receive a passive fiducial; and
a cable management feature configured to receive cables of the histotripsy therapy transducer and prevent the cables from obscuring the passive fiducial.
